(19) United States
(12) Patent Application Publication
(10) Pub. No.: US 2004/0143012 A1
(43) Pub. Date: Jul. 22, 2004

(54) Method for stabilising particle size distribution of powder active principle dispersed in a liquid and uses thereof

(76) Inventors: Rene Gimet, Valbonne (FR); Claude Laruelle, Villeneuve-Loubet (FR); Dominique Toselli, Nice (FR)

Correspondence Address: Welsh & Katz, 22nd Floor, 120 S Riverside Plaza, Chicago, IL, 60606, US

(21) Appl. No.: 10/257,530

(22) PCT Filed: Apr. 11, 2001
(86) PCT No.: PCT/FR01/01122

(30) Foreign Application Priority Data
Apr. 12, 2000 (FR) 00/04679

(51) Int. Cl. A61K 31/203

(52) U.S. Cl. 514/559

(57) ABSTRACT

The invention concerns a method for stabilising particle size distribution of a powdery active principle, when the latter is dispersed in a liquid. Said method is characterised in that it consists in dispersing said active principle in a glycol ether or a mixture of glycol ethers. The invention also concerns the uses of said method and, in particular for pharmaceutical compositions and galenic forms comprising a retinoid as active principle, and suited for providing the latter with improved bioavailability (suprabioavailability) after oral administration.

The present invention relates to a process for stabilizing the particle size of a pulverulent active principle when it is dispersed in a liquid, and also to the uses of this process and especially to pharmaceutical compositions and presentation forms comprising a retinoid as active principle, which are capable of giving this active principle improved bioavailability (“superbioavailability”) after oral administration.

Certain retinoids derived from vitamin A, among which may be mentioned isotretinoin (INN), and acitretin (INN), have been used orally for fifteen years for the treatment of dermatological complaints in their severe forms, for instance nodulocystic acne and acne conglobata, acnes that are resistant to a conventional treatment (antibiotic therapy+topical care) of at least 3 months, serious cases of psoriasis and dermatoses associated with major keratinization disorders (severe ichthyosis, Darier's disease, palmoplantar keratoderma, etc.).

More recently, it has been proposed to use retinoids derived from vitamin A, again orally, for the treatment of cancer pathologies, due to the fact that these compounds have the capacity to block cell proliferation and differentiation by means of interaction with specific receptors known as retinoic acid receptors. Thus, since 1996, French hospitals have had available a specialty product that contains tretinoin (INN) as active principle, for inducing, in combination with conventional chemotherapy, remission in the case of acute promyelocytic leukemia.

Pharmaceutical specialty products currently available in France for the oral administration of isotretinoin (Roaccutane® 5, 10 and 20 mg) and tretinoin (Vesanoid® 10 mg) are in the form of soft capsules in which these active principles are in suspension in an oily vehicle composed of yellow wax, nonhydrogenated and hydrogenated soybean oils and partially hydrogenated plant oils. The recommended dosage is 0.5 to 1 mg/kg/day in the case of isotretinoin, ie a daily intake of 3 capsules of Roaccutane® 10 mg to 3 capsules of Roaccutane® 20 mg for a 60 kg adult, whereas it is 45 mg/m² of body surface and per day in the case of tretinoin, which corresponds to a daily intake of 8 capsules of Vesanoid® 10 mg for a 60 kg adult. These capsules, the preparation of which is described in French patent No. 71/22860, give the active principles they contain relatively poor bioavailability; thus, for example, the bioavailability of isotretinoin is about 25%.

As regards acitretin, it is presented in the form of gel capsules containing a 10 and 25 mg dose and in which this active principle is combined with gelatin, maltodextrin, microcrystalline cellulose and sodium ascorbate. In this case also, these gel capsules afford acitretin limited bioavailability, since it is on average 60%.

However, all these retinoids have many pronounced, dose-dependent adverse effects, some of which are potentially serious, for instance metabolic, hepatic and renal disorders (seric elevation in triglycerides, cholesterol, transaminases, creatinine, seric decrease in HDLs, etc.), and which justify the development of formulations capable of increasing its bioavailability, so as to obtain a better therapeutic efficacy/dose response.

Thus, European patent application No. 0 184 942 proposes to prepare an isotretinoin composition with improved bioavailability by dispersing this active principle in a mixture of excipients comprising a suspension agent preferably consisting of waxes, an antioxidant for stabilizing the isotretinoin, such as butylated hydroxyanisole (BHA) or propyl gallate, a complexing agent, for instance disodium edetate, and also a vehicle composed of a plant oil of the type such as groundnut oil, soybean oil or sesame oil, and by reducing the particle size of the isotretinoin, before it is incorporated into these excipients, such that it is in the form of particles measuring less than 12 μm and preferably less than 10 μm on average.

Specifically, it is well known that the micronization of an active principle has the effect of increasing its area of contact with the biological liquids with which it comes into contact after ingestion, and is reflected in principle, especially in the case where the active principle is sparingly hydrophilic, by an increase in its rate of dissolution and, consequently, in its bioavailability.

Isotretinoin is a compound that is highly sensitive to light and to oxygen, like all vitamin A derivatives. Thus, any production of a composition involving a preliminary mechanical reduction of the particle size of isotretinoin, as proposed in European patent application No. 0 184 942, requires the availability of relatively complex and expensive industrial installations in order to avoid a degradation of this active principle during this reduction.

International PCT patent application No. WO 98/16204 describes a process for obtaining an active principle, and more particularly a carotenoid such as β-carotene, in the form of a powder whose particles are less than 10 μm or even less than 1 μm in size, and in doing so without resorting to a micronization of this active principle. This process consists in dissolving said active principle in dimethyl ether inside a preheated chamber subjected to high pressure (2×10⁷ Pa in the case of isotretinoin), then in suddenly decompressing the resulting solution in a second chamber, for example by spraying, and in separating out the micropowder of active principle thus obtained from the dimethyl ether, which itself is released in gaseous form. In this case also, specific, relatively complex and expensive installations are required to carry out this process.

Continuing their investigations into improving bioavailability of active principles, the Inventors have found that the particle size of an isotretinoin powder increases drastically when this powder is dispersed in a liquid and especially in plant oils, for instance those present in the compositions described in French patent No. 71/22860 and in European patent application No. 0 184 942. Thus, for example, an isotretinoin powder with an initial median particle size of 20 μm has, after dispersion in soybean oil, a particle size whose median is about 100 μm.

These findings are consistent with the observations reported by G. Boullay in his article “Microbroyage et dissolution [Microgrinding and dissolution]” (STP Pharma, 1985, 1, 4, 296-299). Specifically, said author shows that the particles resulting from the micronization of an active principle, once dispersed in a liquid, have a tendency to clump together by the action of electromagnetic forces and thus to form aggregates that considerably reduce the benefit theoretically provided by the micronization, since the area offered for dissolution corresponds to the outer surface area of these aggregates rather than that of the particles.

Thus, the use of techniques for converting isotretinoin into microfine powder, as proposed in European patent application No. 0 184 942 and international PCT application No. WO 98/16024, appears to be of limited interest, since the particle size of this powder is not conserved when the isotretinoin is subsequently dispersed in liquid excipients for the production of a pharmaceutical composition.

The Inventors thus set themselves the aim of overcoming this problem and of providing a process that avoids, when a pulverulent active principle is dispersed in a liquid, the clumping together of the particles of this active principle and the formation, when combined, of aggregates that are liable to halt the subsequent dissolution of said active principle in biological media and consequently reduce its bioavailability.

The Inventors have furthermore set themselves the aim of providing such a process that is very easy to carry out and that can be performed using apparatus with which pharmaceutical laboratories are conventionally equipped for the manufacture of liquid presentation forms.

These aims are achieved according to the invention by a process for stabilizing the particle size of a pulverulent active principle when this active principle is dispersed in a liquid, characterized in that it consists in dispersing this active principle in a glycol ether or a mixture of glycol ethers.

Glycol ethers are a family of amphiphilic (ie both hydrophilic and lipophilic) solvents, derived from ethylene glycol and propylene glycol, which correspond to formulae (I) and (II) below:

R¹—[O—CH₂—CH₂]ₙ—O—R² (I)

R¹—[O—CH₂—CH(CH₃)]ₙ—O—R² (II)

in which

n is equal to 1, 2 or 3,

R¹ represents a linear or branched alkyl group, generally of C₁ to C₆, whereas

R² represents a hydrogen atom or a group C(O)—R³ in which R³ usually represents a methyl group (in which case they are referred to as glycol ethers-esters).

According to a first advantageous embodiment of the invention, the glycol ether(s) is (are) chosen from ethylene glycol derivatives, also known as glycol ethers of the E series. Examples of such ethers that may be mentioned include ethylene glycol monomethyl, monoethyl, monopropyl and monobutyl ethers, diethylene glycol monomethyl, monoethyl, monopropyl and monobutyl ethers, ethylene glycol dimethyl and diethyl ethers, diethylene glycol dimethyl and diethyl esters, triethylene glycol monomethyl, monoethyl and monobutyl ethers, ethylene glycol monomethyl ether acetate, diethylene glycol monomethyl ether acetate and diethylene glycol monoethyl ether acetate.

As a variant, it is possible to use one or more ethers derived from propylene glycol (or glycol ethers of the P series), for instance propylene glycol monomethyl, monoethyl and monobutyl ethers.

According to one preferred embodiment of the invention, the active principle is dispersed in diethylene glycol monoethyl ether (DGME), also known as ethyl diglycol.

The use of this compound as a pharmaceutical excipient is not novel per se. Specifically, it has already been proposed, in French patent application No. 95/09142 in the name of the Applicant, to use DGME as a fenofibrate solubilizer, its ability to dissolve said active principle being found, in point of fact, to be very much greater than that presented by the solvents conventionally used for lipophilic molecules, for instance propylene glycol, glycerol and polyoxyethylene glycols (PEG 200, 300, 400, etc.).

What is, however, entirely novel is the finding made by the Inventors regarding the property shown by DGME, just like the other members of the family of glycol ethers, to allow a pulverulent active principle to conserve, after dispersion in one of these glycol ethers, the particle size that it has in dry form.

According to another advantageous embodiment of the invention, the active principle/glycol ether(s) ratio is between 0.01 and 0.09 (w/v calculated in kg/l).

In a particularly advantageous manner, the active principle is a retinoid.

For the purposes of the present invention, the term “retinoid” means any compound capable of binding to and interacting with a retinoic acid receptor (RAR alpha, beta or gamma) or to a retinoid X receptor (RXR alpha, beta or gamma). Examples of such retinoids that may be mentioned include, firstly, retinoids derived from vitamin A, for instance tretinoin, also known as all-trans-retinoic acid or all-trans-vitamin A acid, isotretinoin, which corresponds to the 13-cis isomer of tretinoin and which is accordingly also known as 13-cis-retinoic acid or 13-cis-vitamin A acid, 9-cis-retinoic acid or 9-cis-vitamin A acid, acitretin and etretinate, and also acetylenic retinoids, for instance tazarotene, naphthalene-based retinoids, for instance lonapalene and 2-(5,6,7,8-tetrahydromethyl-2-anthryl)-4-thiophenocarboxylic acid, and retinoids containing an adamantyl ring, such as adapalene, 6-[3-(1-adamantyl)-4-hydroxyphenyl]-2-naphthoic acid and 4-[3-(1-adamantyl)-4-methoxybenzamido]benzoic acid, and esters thereof.

The retinoid is preferably isotretinoin.

The process for stabilizing the particle size of a pulverulent active principle in accordance with the invention has many advantages. Specifically, besides the fact that it is extremely simple to carry out and does not require any specific equipment since it suffices to disperse the active principle whose particle size it is desired to stabilize in a glycol ether or a mixture of glycol ethers in order to obtain the desired result, it allows a real benefit to be obtained from the use of an active principle that is in the form of a microfine powder, and thus of techniques for obtaining such a powder, for instance micronization.

Moreover, this process makes it possible to prepare, from pulverulent active principles as are commercially available, pharmaceutical compositions capable of giving these active principles better bioavailability after oral administration, and to do so without it being necessary to resort to any operation intended to reduce their particle size. Thus, when applied to the preparation of pharmaceutical compositions intended to contain fragile active principles, for instance isotretinoin, tretinoin or acitretin, it allows a considerable reduction in the risk of degradation of these active principles during this preparation and also in the cost price of said compositions.

A subject of the present invention is thus also a process for improving the bioavailability of a retinoid when it is administered orally, said process being characterized in that it comprises the formulation of this retinoid in a presentation form that is suitable for oral administration and in which said retinoid is present in the form of particles suspended in a glycol ether or a mixture of glycol ethers.

A subject of the present invention is also a pharmaceutical composition for oral administration, which is characterized in that it contains an effective amount of particles of a retinoid suspended in a glycol ether or a mixture of glycol ethers.

According to a first advantageous embodiment of this composition, the glycol ether(s) is (are) chosen from diethylene glycol ethers.

Preferably, the particles of the retinoid are suspended in diethylene glycol monoethyl ether.

According to another advantageous embodiment of this composition, the retinoid/glycol ether(s) ratio is between 0.01 and 0.09 (w/v calculated in kg/l).

According to one particularly preferred embodiment of this composition, 50% by weight of the retinoid particles it contains are between 15 and 40 μm in diameter and are preferably substantially equal to 20 μm.

In accordance with the invention, the pharmaceutical composition may comprise at least one or more other excipients such as, for example, antioxidants, preserving agents or agents for modifying the viscosity of this composition.

The retinoid is preferably isotretinoin.

A subject of the present invention is also a presentation form of a retinoid for oral administration, which is characterized in that it contains a pharmaceutical composition as defined above in a soft capsule, preferably made of gelatin.

Advantageously, each capsule contains a dose of isotretinoin of between 1 and 35 mg.

Such a presentation form may be prepared by a process comprising:

the dispersion of the retinoid in a glycol ether or a salt of such an ether until a uniform suspension is obtained, followed by the incorporation of the suspension thus obtained into soft capsules.

The invention will be understood more clearly with the aid of the rest of the description which follows, which refers to comparative studies regarding the particle size of an isotretinoin powder before and after dispersion in various liquid media, including DGME, and to an example of the preparation of a presentation form of isotretinoin in accordance with the invention, and also to the attached plate of drawings.

A more detailed explanation of the invention is provided in the following description and claims take in conjunction with the accompanying drawings.

### BRIEF DESCRIPTION OF THE DRAWINGS

FIG. 1 illustrates the particle size presented by the isotretinoin powder before dispersion in a liquid medium;

FIG. 2 illustrates the particle size of the isotretinoin powder after dispersion in nonhydrogenated soybean oil;

FIG. 3 illustrates the particle size of the isotretinoin powder after dispersion in a mixture composed of yellow wax, nonhydrogenated and hydrogenated soybean oils, a partially hydrogenated plant oil and a medium-chain triglyceride;

FIG. 4 illustrates the particle size of the isotretinoin powder after dispersion in caprylocapric macrogolglycerides;

FIG. 5 illustrates the particle size of the isotretinoin powder after dispersion in propylene glycol dicaprylocaprate; whereas

FIG. 6 illustrates the particle size of the isotretinoin powder after dispersion in DGME.

It goes without saying, however, that the remainder of the description is given purely by way of illustration of the invention and does not in any way constitute a limitation thereof.

### EXAMPLE 1

Comparative studies regarding the particle size of an isotretinoin powder after dispersion in various liquid media

The ability of glycol ethers to stabilize the particle size of an active principle was established by means of studies aimed at comparing the particle size of an isotretinoin powder supposed to have, according to its supplier, a median of 20 μm, before and after dispersion in 5 different liquid media consisting, respectively, of:

nonhydrogenated soybean oil,

an oily mixture of the type present in Roaccutane® capsules and composed of yellow wax, nonhydrogenated and hydrogenated soybean oils, a partially hydrogenated plant oil and a medium-chain triglyceride,

of caprylocapric macrogolglycerides (mixture of polyethylene glycol mono- and diesters and mono-, di- and triglycerides, sold under the brand name Labrasol®—Gattefosse company),

propylene glycol dicaprylocaprate (Labrafac® PG—Gattefosse company), and

diethylene glycol monoethyl ether or DGME (Transcutol®—Gattefosse company).

The particle size of the isotretinoin powder before dispersion in the liquid media, ie in dry form, was analyzed on samples of 2 g of powder (the vehicle in this case being compressed air), whereas its particle size after dispersion in the various liquid media was analyzed on samples of 20 ml containing 2% (w/v) of this powder, except in the case of the oily mixture, in which the content of isotretinoin powder in the samples was 6.5% (w/v).

All the particle size analyses were carried out using a Mastersizer Banc Long laser scattering granulometer (Malvern Instruments Ltd company) and an MS1 sampler from the same company.

FIGS. 1 to 6 illustrate, both in the form of histograms and curves, the particle size distributions obtained for the isotretinoin powder, respectively:

in dry form (FIG. 1),

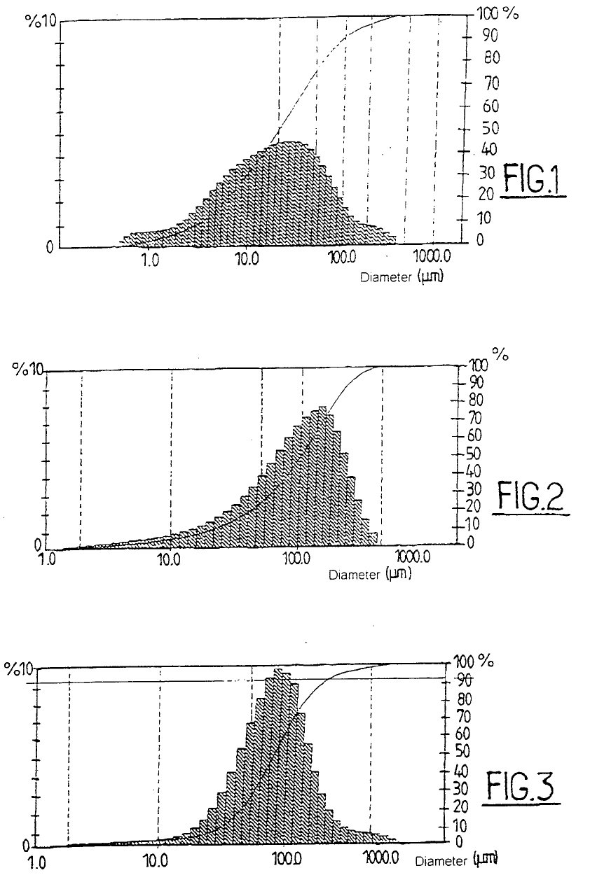

after dispersion in hydrogenated soybean oil (FIG. 2),

after dispersion in the oily mixture (FIG. 3),

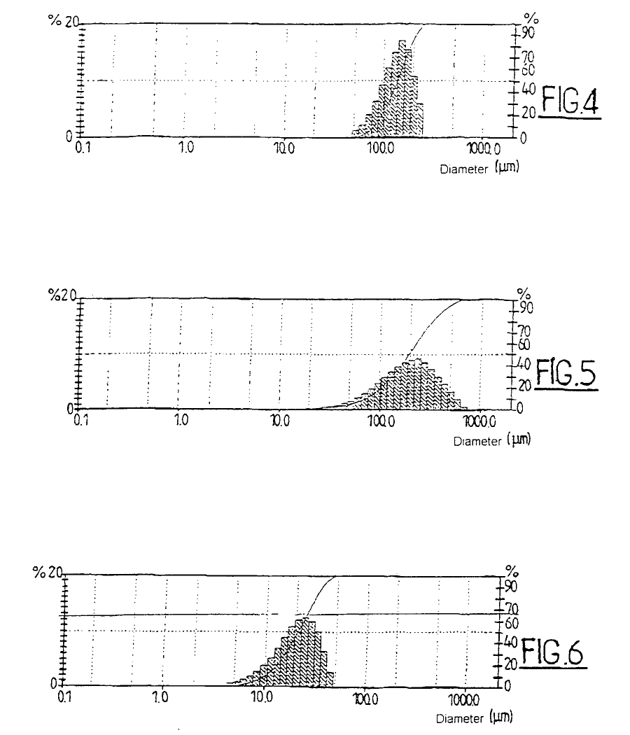

after dispersion in Labrasol® (FIG. 4),

after dispersion in Labrafac® PG (FIG. 5), and

after dispersion in Transcutol® (FIG. 6).

In FIGS. 1 to 6, the x-axes represent the diameter, expressed in μm, of the isotretinoin particles, the left-hand y-axes represent the percentage of each population of isotretinoin particles - a population corresponding to a shaded rectangle -, whereas the right-hand y-axes represent the cumulative percentages of the populations of said particles.

Table 1 below shows the medians of the particle sizes, expressed in Jim, presented by the isotretinoin powder.

TABLE 1

| Media | Medians (μm) |
|---|---|
| dry powder | 19.49 |
| nonhydrogenated soybean oil | 103.74 |
| oily mixture | 82.92 |
| Labrasol 200 | 139.77 |
| Labrafac ® PG | 193.15 |
| Transcutol ® | 20.86 |

The analyses performed on the isotretinoin powder in dry form confirmed that it does indeed have a median particle size substantially equal to 20 μm, as stated by its supplier.

Moreover, the analyses performed on this same powder after dispersion in the 5 liquid media tested show that its particle size increases drastically (by a factor of 4 to 10) in the standard oily vehicles (soybean oil, oily mixture), like in the vehicles more recently commercialized (Labrasol®, Labrafac® PG), except in a glycol ether, for instance diethylene glycol monoethyl ether.

### EXAMPLE 2

Preparation of soft capsules containing 5 mg of isotretinoin in DGME

100 000 soft opaque gelatin capsules containing a 5 mg dose of isotretinoin are prepared by dispersing, in a Fryma mixer, under vacuum and protected from light, 0.5 kg of an isotretinoin powder as used in Example 1 in 25 liters of Transcutol® until a uniform suspension is obtained, followed by transferring this suspension into a Swisscaps encapsulating machine.

The soft capsules thus prepared each contain 5 mg of isotretinoin with a median particle size substantially equal to 20 μm in 0.5 ml of DGME.

By comparison, a particle size analysis performed on 20 ml samples corresponding to the content of 25 Roaccutane® capsules containing a 5 mg dose revealed that the isotretinoin contained in these capsules has a median particle size of between 80 and 130 μm.

1. A process for stabilizing the particle size of a pulverulent active principle when this active principle is dispersed in a liquid, characterized in that it consists in dispersing this active principle in a glycol ether or a mixture of glycol ethers.

2. The process as claimed in claim 1, characterized in that the glycol ether(s) is (are) chosen from diethylene glycol ethers.

3. The process as claimed in claim 1 or claim 2, characterized in that the active principle is dispersed in diethylene glycol monoethyl ether (DGME).

4. The process as claimed in any one of the preceding claims, characterized in that the active principle/glycol ether(s) ratio is between 0.01 and 0.09 (w/v).

5. The process as claimed in any one of the preceding claims, characterized in that the active principle is a retinoid.

6. The process as claimed in any one of the preceding claims, characterized in that the active principle is isotretinoin.

7. A process for improving the bioavailability of a retinoid when it is administered orally, characterized in that it comprises the formulation of this retinoid in a presentation form that is suitable for oral administration and in which said retinoid is present in the form of particles suspended in a glycol ether or a mixture of glycol ethers.

8. A pharmaceutical composition for oral administration, characterized in that it contains an effective amount of particles of a retinoid suspended in a glycol ether or a mixture of glycol ethers.

9. The pharmaceutical composition as claimed in claim 8, characterized in that the glycol ether(s) is (are) chosen from diethylene glycol ethers.

10. The pharmaceutical composition as claimed in claim 8 or claim 9, characterized in that the particles of the retinoid are suspended in diethylene glycol monoethyl ether (DGME).

11. The pharmaceutical composition as claimed in any one of claims 8 to 10, characterized in that the retinoid/glycol ether(s) ratio is between 0.01 and 0.09 (w/v).

12. The pharmaceutical composition as claimed in any one of claims 8 to 11, characterized in that 50% by weight of the retinoid particles are between 15 and 40 μm in diameter and are preferably substantially equal to 20 μm.

13. The pharmaceutical composition as claimed in any one of claims 8 to 12, characterized in that it furthermore comprises one or more other excipients chosen from antioxidants, preserving agents and agents for modifying the viscosity of this composition.

14. The pharmaceutical composition as claimed in any one of claims 8 to 13, characterized in that the retinoid is isotretinoin.

15. A presentation form of a retinoid for oral administration, characterized in that it contains a pharmaceutical composition as claimed in any one of claims 8 to 14 in a soft capsule, preferably made of gelatin.

16. The presentation form as claimed in claim 15, characterized in that each capsule contains a dose of isotretinoin of between 1 and 35 mg.
